(19) United States
(12) Patent Application Publication
(10) Pub. No.: US 2007/0017840 A1
(43) Pub. Date: Jan. 25, 2007

(54) Stacking of Fibrous Pads

(75) Inventors: Bernard Louis Dit Picard, 27370 Amfreville La Campagne (FR); Philippe Gregoire, 27700 Les Andelys (FR)

Correspondence Address: PATENT GROUP GA030-43;GEORGIA-PACIFIC CORPORATION, 133 PEACHTREE STREET, N.E., ATLANTA, GA, 30303-1847, US

(73) Assignee: GEORGIA-PACIFIC FRANCE, Kunheim (FR)

(21) Appl. No.: 11/457,846

(22) Filed: Jul. 17, 2006

(30) Foreign Application Priority Data
Jul. 22, 2005 (FR) 05 07794

(51) Int. Cl. A61B 19/02

(52) U.S. Cl. 206/440

(57) ABSTRACT

The object of the invention is a stacking of fibrous pads, in particular impregnated pads, for skin care, capable of being contained in packaging. According to the invention, each pad is positioned in a staggered manner in relation to the juxtaposed pad(s) so as to promote the grasping by one of the edges of each pad situated on the top of the stack. The invention also relates to resealable packaging containing the stacking as well as to a manufacturing process for preparing the stacking.

This invention relates to the field of products for cosmetic use or for skin care, specifically to a stack of pads made of a fibrous material, preferably impregnated with a lotion to be applied to the skin, for example, for the purpose of make-up removal, cosmetic care, cleansing of the skin, and the cleaning of infants in particular.

For the purpose of make-up removal, use is generally made of a pad of cotton or other fibrous material on which a small amount of lotion or make-up removal liquid is deposited which is then rubbed onto the skin so as to dilute or dissolve the make-up or impurities which are then peeled away or absorbed by the cotton.

Pads, also termed “formats”, are currently available and marketed in a large number of shapes. They are cut out of a web of nonwoven fibrous material, especially material made of natural fibers, such as cotton fibers. The pad may also incorporate synthetic and/or artificial fibers, such as viscose rayon fibers.

The pads exist in a wide variety of sizes, from less than 25 cm² to more than 100 cm², that are circular, oval, square, rectangular, etc., in shape and have grammages from approximately 100 to approximately 300 g/m², preferably from 180 to 250 g/m².

A fibrous pad intended for this application must perform several functions:

It must first absorb the lotion or the liquid, if possible not too deeply so that this lotion and this liquid may remain accessible on the surface.

It must release this lotion or this liquid when the lotion or liquid is pressed against the skin in order to dilute or dissolve the make-up or impurities.

It must absorb and wipe the diluted or dissolved make-up so as to leave the skin neat and clean.

It must be thick enough so that it can be held firmly in the hand during use without falling apart.

The pad must also remain whole during use. It should not shed any fluff nor leave any fibers on the skin. It should retain its shape and not fall apart in any of its three dimensions while being handled. This strength characteristic is measured in its three dimensions: machine direction (SM) strength, in the running direction of the nonwoven web; cross direction (ST) strength, perpendicular to the machine direction (SM), and decohesion force (D), which is the force of separation of the two pad surfaces in the thickness direction.

It should be noted that the mechanical properties of pads have been improved in recent years over those of the plain carded cotton web from which the pads were originally made, by application of either of the two following processes:

Incorporation into the mass of fibers of a fusible bonding agent (in the form of fibers or a powder), accompanied by heating with hot air or hot calendering. The bonding agent joins together the cotton fibers during fusion followed by cooling and makes it possible to increase the strength of the pads in their three dimensions. This process, however, is not applicable for products that are intended to be made exclusively of cellulose fibers.

Treatment of the fiber web by means of water jets, according to a hydroentangling process that entangles the surface fibers. This process makes it possible to reduce the tendency toward fluffiness and to increase the strength of the web. This purely mechanical process makes it possible to manufacture webs made of 100% cotton.

Another prior-art means of make-up removal consists in the use of pads already impregnated with a make-up removal solution.

The impregnation of a pad may be defined as the ratio of the weight of the added lotion to the weight of the fibrous support. This ratio is commonly between 1 and 6, preferably between 2 and 5.

Such impregnated products are available commercially and are usually packaged in a stack in an impervious box made of plastic. They may also be packaged in a plastic bag or sealed flexible film.

As these pads are impregnated with a lotion for make-up removal or for skin care, they are therefore ready to use.

Owing to their practicality, they advantageously replace the set comprising a dry cotton pad and a flask of lotion or liquid.

The impregnated cotton pads must be packaged in flexible or rigid packaging so as to make it possible to avoid a noticeable evaporation of the lotion and/or a bacterial contamination. For the same reasons, the packaging products known in the prior art have an opening/closing system for putting them into service, which system is useable throughout the period of their utilization.

The problem encountered with stacks of conventional cotton pads, particularly the impregnated ones, lies in that the user must be able to lift up each pad individually, easily and without risk of decohesion.

This problem of decohesion is significant because, as the only accessible pad is the one on top of the stack, the lifting up of a pad can be accomplished conventionally only by pinching the top surface of the pad. This manner of proceeding is not always satisfactory and numerous incidents may take place when the pads are lifted up: the user either involuntarily lifts up several pads at once, or picks up only a part of one pad (the pad having separated in the direction of its thickness if the force of decohesion is too weak). In the latter case, the pad delaminates. This is relatively frequent for pads of a certain grammage, made from spunlaced web whose fibers are essentially surface-bonded.

One way to solve this problem consists in, as taught by French Patent Application EN 05 50424, cotton pads composed of a fibrous substrate made comprising cotton fibers, impregnated with a lotion to be applied to the skin, according to which the substrate is a nonwoven capable of releasing under pressure at least 250 g/m² of lotion, with the dry substrate having a decohesion force of at least 2.5 N and a thickness of at least 1.2 mm.

The intrinsic and combined characteristics of these pads are, namely, their ability to release lotion, their dry cohesion force, and their thickness allowing the problem of decohesion to be remedied and a particularly low rate of decohesion to be achieved.

However, that solution is particular and specific for pads not having the requisite characteristics will still present decohesion problems.

This invention as claimed proposes a different and original solution, which applies to a much wider range of fibrous, preferably impregnated, pads.

Also known from the prior art is US2004/0245139, which describes packaging for moist pads inside which the pads are arranged in two stacks having an overlapping of space. To achieve this, the packaging presents a particular shape.

This type of packaging is rather costly and bulky (almost two juxtaposed stacks).

This invention proposes a solution that is at once simple, effective, reliable, attractive and low cost, that permits a lifting up of the stacked pads without placing any constraints on the characteristics of the fibrous substrate itself.

More specifically, the invention relates to an arrangement of superposed fibrous pads, permitting an easy lifting up of the upper pads (at the top end of the stack).

Thus, the object of the invention is a stacking of fibrous pads for make-up removal and/or skin care, capable of being contained in packaging.

Preferably, the fibrous pads are impregnated.

According to the invention, each pad is positioned in a staggered manner in relation to the superposed pad(s) in order to promote the grasping of the pad situated at the top of the stack by one of its edges.

Thus, the lifting up of the pad is possible by way of an edge (border) of the pad, which allows its two sides to be pinched simultaneously and therefore facilitates its lifting up without the risk of decohesion of the pad.

In addition, the stacking is easy to achieve and is non-bulky.

According to the invention, the staggering between two juxtaposed pads may be angular and/or may be in relation to at least one of the dimensions of the pad.

Angular staggering is preferred for pads with a non-circular main surface.

Preferably, the angular staggering between two consecutive pads can be between 1° and 179°, preferably between 2° and 90°.

It is therefore possible to achieve stackings that are both balanced and aesthetically pleasing.

In another embodiment, the staggering in relation to the at least one dimension comprises between 2 and 60 mm, preferably between 5 and 20 mm.

Thus, by staggering in at least one dimension each pad from the pad directly juxtaposed to or superposed on it, at least a part of each edge does not coincide with the edge of the juxtaposed pad. The grasping of the pad at the top of the stack (by an edge) is thereby facilitated and the risk of grasping all or part of the juxtaposed pad is thus avoided.

Preferably, the pads are made mostly of cotton fibers.

The invention preferably relates to resealable packaging containing a stack of impregnated fibrous pads such as that defined above.

The invention also includes a manufacturing process for preparing a stack of impregnated fibrous pads comprising in particularly the following stages including:

unwinding a strip of nonwoven fibrous material;

cutting out pads (or formats) from this strip;

separating the pads from the rest of the strip;

conveying the pads to a stacking station.

According to the invention, the stacking includes wedging each pad against a thrust block, then in depositing the pad on the stack already formed or onto the bottom of the packaging, then displacing the thrust block in order to modify the position of the next pad before it is deposited onto the stack.

Thus, the thrust block changes its position before wedging the next pad into a staggered position in relation to that of the preceding pad.

According to another embodiment of the invention, the stacking includes wedging each pad against a thrust block, then depositing the pad onto a receptacle, then causing the receptacle to rotate so as to obtain an angular staggering of the pad in relation to the next pad.

The invention will be better understood through the description that follows which is given solely for illustrative purposes and is in no way limitative, with reference to the attached drawings, in which:

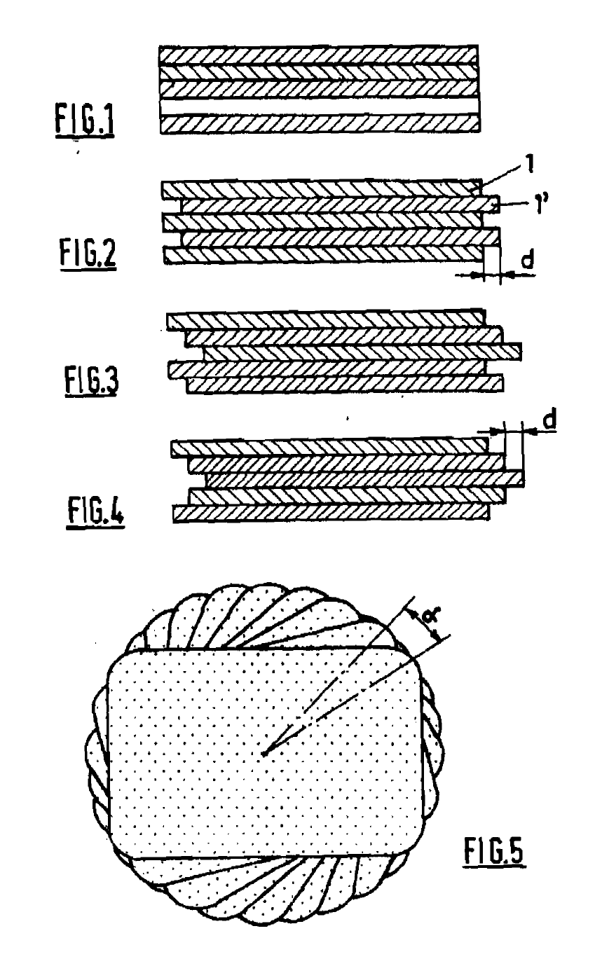

FIG. 1 is a cross-section of a conventional stacking of pads;

FIGS. 2, 3, and 4 are cross-sections of several possible stackings according to the invention; and

FIG. 5 is an overhead view of a stacking according to one embodiment of the invention.

According to FIG. 1, the pads (or formats) are stacked so that their surfaces coincide totally, which makes the pads difficult to grasp by their edges, resulting in the problems detailed above, in particular in the decohesion of the impregnated pads.

In a novel and inventive manner, the stacking is achieved according to one of the following embodiments.

According to FIG. 2, a regular staggering is achieved through an alternation by twos, whereas FIG. 3 shows a staggering through alternation by threes.

Alternatively, in another embodiment, FIG. 4 shows a staggering involving a somewhat slightly different sequence that is entirely understandable from the cross-section shown in FIG. 4 itself.

In all cases, the staggering between two juxtaposed pads 1, 1′ is preferably on the order of between 2 and 60 mm, preferably between 5 and 20 mm.

Here, staggering d, is understood to mean the maximum distance “from edge to edge” of the two superposed stacked pads, as measured in a single direction of the main plane.

FIG. 5 illustrates an example of a particular stacking according to which each pad is staggered angularly at a certain angle in relation to the superposed pad.

The staggering angle “α” is measured according to the main axis of the stacking.

Thus, according to the case shown in the FIGURES, either the impregnated pads are oriented in the same manner (FIGS. 2 to 4), in which these cases the staggering is measured by the predefined distance “d”, or the pads are not oriented in the same manner in the stack, and the staggering is then measured by the angle “α” between two pads, measured according to the axis of the stacking.

Depending upon the individual form of each pad (which may be any shape), on the geometry, if not the final aspect, of the stacking, and on the type of packaging, then one or the other, or even a combination of these staggerings, will be preferred.

When a completely hermetic packaging is desired, a rigid cylindrical packaging is preferably selected because it is then possible to adapt a circular cover, for example, one screwed onto the top of the packaging. In this case, a stacking as claimed by the invention will be formed, which stacking has an outer shape that is overall cylindrical with an angular staggering. FIG. 5 is an illustration of such a stacking.

By way of illustration, two embodiments of the invention will now be described:

### EXAMPLE 1

A reel of nonwoven web is unwound from which is cut, by any known means, pads in a selected shape. The cutting can be done conventionally on the basis of rotation. In this case, the web passes continuously between two cylinders, one with cutting tools jutting out and the other being smooth.

The cutting may also be done by means of alternation, with the web advancing step by step between the two parts of a cutting die.

The pads are then separated from the trimmings by various means known in the prior art, for example, a reciprocating piston ejector or a cylinder or a suction conveyor, or a deviation from the run of the trimmings.

The pads are then conveyed to a stacking station. This station may include a reciprocating piston plunger allowing the lifting up of each pad from the conveyor and the stacking of the pads into a receptacle situated either above or under this station. The conveyor of the individual pads is outfitted with cut-outs that allow for the passage of the pistons.

The conveyor wedges the pad against a thrust block, then the reciprocating piston plunger transfers the pad into the receptacle. The staggering of a pad in relation to the preceding pad may be carried out by a displacement of the thrust block, which modifies the wedging position of the pad, just before the plunging of the piston.

By this means and according to the positioning sequence of the thrust block, the stackings can be achieved as shown in FIGS. 2 to 4.

### EXAMPLE 2

A roll of nonwoven web is unwound, in which pads are cut by any prior art means in a selected shape. Conventionally, the cutting process may be rotational. In this case, the web passes continuously between two cylinders, one with the cutting tools projecting out and the other smooth.

The cutting process may also be done on the basis of alternation, with the web advancing step by step between the two parts of a cutting die.

The pads are then separated from the trimmings, using various means, such as, for example, a reciprocating piston ejector or a cylinder or a suction conveyor or by means of deviating them from the path of the trimmings.

The pads are then conveyed to the stacking station. This station may include a reciprocating piston plunger allowing the lifting up of each pad from the conveyer and the stacking of the pads into a receptacle situated either above or under this station. The conveyor of the individual pads is outfitted with cut-outs that allow the passage of the pistons.

The receptacle is rotatable and pivots a few degrees between each pushing of a pad. By this means an angular staggering is obtained between each pad in relation to the preceding pad and a stacking is carried out as shown FIG. 5. Preferably, a stationary thrust block is inserted on the pad conveyor in order to properly adjust the position of the pads before they are pushed by the piston toward the rotating receptacle.

Of course the embodiments of the invention are not limited to those described above.

In particular, pad impregnation may be accomplished on each individual pad, between the cutting station and the stacking, or it may be accomplished directly on the stacking already formed, before or during the packaging stage.

What is claimed is:

1. A stack of fibrous pads for make-up removal and skin care, capable of being contained in packaging, comprising: a plurality of fibrous pads wherein each pad is positioned in a staggered manner in relation to one or more superposed pads in order to promote grasping of one edge of a pad situated at the top of the stack.

2. The stack according to claim 1, wherein at least one of the fibrous pads is impregnated with a fluid.

3. The stack according to claim 1, wherein each pad is staggered in relation to at least one superposed pad according to at least one dimension of the pad.

4. The stack according to claim 3, wherein the at least one dimension is between 2 and 60 mm.

5. The stack according to claim 1, wherein each pad is staggered in relation to at least one superposed pad at an angle to the at least one superposed pad.

6. The stacking according to claim 5, wherein the angle is between 1° and 179° measured along a main axis of the stack.

7. The stack according to claim 1, wherein said fibrous pads are composed of substantially cotton fibers.

8. The stack according to claim 1, wherein the stack is contained in resealable packaging.

9. A resealable packaging containing a stack of fibrous pads according to any one of claims 1 to 8.

10. A process for manufacturing a stack of fibrous pads comprising:
unwinding a strip of nonwoven fibrous material;
cutting pads out of the strip;
separating the pads cut from the strip;
transporting the pads to a stacking station;
stacking the pads to form a stack by wedging each pad against a thrust block,
depositing the pad on the stack already formed on a bottom of packaging, and displacing the thrust block in order to change position of a next pad before depositing the next pad onto the stack already formed.

11. A process for manufacturing a stack of fibrous pads comprising:
unwinding a sheet of nonwoven fibrous material;
cutting pads out of the sheet;
separating the pads cut from the sheet;
conveying the pads to a stacking station;
stacking the pads to form a stack by wedging each pad against a thrust block, depositing the pad into a receptacle, and causing the receptacle to rotate in order to obtain an angular staggering of the pad in relation to a next pad.
